(19) United States
(12) Patent Application Publication
(10) Pub. No.: US 2015/0297795 A1
(43) Pub. Date: Oct. 22, 2015

(54) CARRIER FOR TRANSFERRING CELL SHEET FOR TRANSPLANTATION

(71) Applicant: NIKKAN INDUSTRIES CO., LTD., Tokyo (JP)

(72) Inventors: Toyoji Hibi, Sakado-shi (JP); Koji Kato, Sakado-shi (JP); Hiroyuki Niikura, Sakado-shi (JP); Jun Watanabe, Tokyo (JP); Masanori Kojima, Tokyo (JP)

(73) Assignee: NIKKAN INDUSTRIES CO., LTD., Tokyo (JP)

(21) Appl. No.: 14/647,335

(22) PCT Filed: Aug. 26, 2013
(86) PCT No.: PCT/JP2013/072703

§ 371 Date: May 26, 2015

(30) Foreign Application Priority Data
Dec. 10, 2012 (JP) 2012-269747

(51) Int. Cl. A61L 27/50; A61L 27/38; B32B 37/18; B32B 9/02; A61L 27/20; A61L 27/18; B32B 5/02; C12N 5/00; B32B 37/12

(52) U.S. Cl. CPC A61L 27/50; C12N 5/0068; A61L 27/38; B32B 37/18; B32B 37/12; A61L 27/20; A61L 27/18; B32B 5/022; B32B 9/02; C12N 2533/70; B32B 2250/02; B32B 2262/0276; B32B 2317/00; B32B 2367/00; B32B 2535/00

(57) ABSTRACT

The object is to provide a carrier showing superior adhesion property and detachability, which is suitable for transferring a cell sheet. A carrier for transferring a cell sheet for transplantation comprising a cell sheet-adhesive coating layer that can respond to a non-cytotoxic condition and a support layer consisting of a base material acceptable as a medical material is provided. By applying a cell sheet adhering to the coating layer of the carrier to a transplantation site and providing the non-cytotoxic condition, the carrier can be detached and removed with maintaining the cell sheet at the transplantation site.

### TECHNICAL FIELD

The present invention relates to a carrier for transferring a cell sheet for transplantation. The present invention is useful in the fields of regenerative medicine, and so forth.

### BACKGROUND ART

When an organ or tissue in the body is lost or causes dysfunction or incompetence, transplantation of cells or tissue is required in order to regenerate the lost function. The medical techniques for rebuilding an organ or tissue function are generically called “regenerative medicine”.

One of the techniques of the regenerative medicine consists of extracting stem cells existing in a tissue in an extremely small amount, culturing them under appropriate conditions in vitro to form a cultured tissue for transplantation, and using the cultured tissue for a treatment of the damaged part. Practical use of such a technique is spreading for such objects as skin epidermis, corneal epidermis, bone, cartilage, and cardiac muscles. Utilization of stem cells derived from iPS cells (induced Pluripotent Stem cells) is also recently much hoped. In order to make these techniques of regenerative medicine widely practical, a method for easily preparing a tissue of high quality for transplantation with sufficient reproducibility is desired for each of the stages of extraction, culture, transplantation of cells, and so forth.

A cell culture for transplantation in the form of a sheet is also called cell sheet. A cell sheet is prepared by culturing stem cells on an appropriate culture vessel to allow proliferation of them, and various researches have been conducted as for surfaces of culture vessels in order to suppress possibility of contamination as well as degeneration and damage of cell sheet at the time of peeling off the cell sheet from the surface of culture vessel. For example, there have been developed techniques for detaching a cell sheet from a surface of culture vessel by coating a surface of a base material with a temperature-responsive polymer, i.e., by culturing cells on such a cell culture support coated with a temperature-responsive polymer to form a sheet, and then adjusting culture medium to an appropriate temperature to dissolve the temperature-responsive polymer and thereby detach the sheet from the surface of the culture vessel (Patent documents 1 to 6).

Further, when a cell sheet detached from a culture vessel is transferred to a transplantation site, the cell sheet is once adhered on a carrier in the form of a film in order to maintain the sheet shape by preventing shrinkage, creasing, sagging, tearing, etc. of the sheet. More precisely, before or after performing the operation for detaching the cell sheet from the surface of the culture vessel, a carrier film consisting of a material to which the cell sheet can adhere is placed on the cultured cell sheet, and the cell sheet is lifted up together with the carrier. Specific examples of the material usable as this carrier include polyvinylidene difluoride (PVDF), polypropylene, polyethylene, cellulose and derivatives thereof, papers, chitin, chitosan, collagen, urethane resins, and so forth (Patent document 4 mentioned above etc.).

### PRIOR ART REFERENCES

### Patent documents

Patent document 1: International Publication WO2006/093153

Patent document 2: International Publication WO2005/103233

Patent document 3: International Publication WO2005/084429

Patent document 4: International Publication WO2004/073761

Patent document 5: International Publication WO02/010349

Patent document 6: International Publication WO02/008387

### SUMMARY OF THE INVENTION

### Object to be Achieved by the Invention

It is desirable that a carrier for transferring a cell sheet can firmly adhere to a cell sheet, and the cell sheet can be lifted up together with the carrier from a surface of culture vessel when the cell sheet is collected from the surface of the culture vessel and transferred to a transplantation site (adhesion property). However, it is preferred that, at the transplantation site, only the carrier can be easily detached and removed with leaving the cell sheet on the surface of affected part (detachability).

However, in fact, only existing food packaging materials etc. have been diverted to such carriers, and any researches have not so far been sufficiently conducted for obtaining a carrier especially suitable for the purpose of transferring a cell sheet.

### Means for Achieving the Object

The inventors of the present invention have examined various materials for suitability for the purpose of transferring a cell sheet. As a result, they found that a polylactic acid nonwoven fabric of which surface was coated with a polymer material mainly consisting of a substance that could dissolve at a comparatively low temperature could show superior adhesion property and detachability. Further, such a carrier is usually handled with a pair of tweezers as a medical tool, and in this respect, they found that such a material as mentioned above could be handled with a pair of tweezers with good handling property, showed less changes of properties due to moisture absorption, and could be finely adhered onto even a spherical surface such as that of eyeball, and accomplished the present invention.

The present invention provides the followings.

[1] A carrier for transferring a cell sheet for transplantation comprising a cell sheet-adhesive coating layer that can respond to a non-cytotoxic condition and a support layer consisting of a base material acceptable as a medical material, wherein:

by applying a cell sheet adhering to the coating layer of the carrier to a transplantation site and providing the non-cytotoxic condition, the carrier can be detached and removed with maintaining the cell sheet at the transplantation site, preferably,

a carrier for transferring a cell sheet for transplantation comprising:

a cell sheet-adhesive coating layer comprising a polysaccharide that can be dissolved, softened, fused, or decomposed, or can show decrease of viscosity at a non-cytotoxic temperature, and

a support layer having the coating layer on a surface thereof and consisting of a nonwoven fabric, woven fabric, knitted fabric, or film of a base material acceptable as a medical material, wherein:

by applying a cell sheet adhering to the coating layer of the carrier to a transplantation site and providing a non-cytotoxic temperature, the carrier can be detached and removed with maintaining the cell sheet at the transplantation site.

[2] The carrier according to [1], wherein the support layer consists of a nonwoven fabric or woven fabric, preferably a polylactic acid nonwoven fabric.
[3] The carrier according to [1] or [2], wherein the coating layer comprises a polysaccharide that can respond to a non-cytotoxic temperature.
[4] The carrier according to [1], wherein the non-cytotoxic condition is provided by dropping an isotonic solution at a non-cytotoxic temperature.
[5] The carrier according to any one of [1] to [4], wherein:

the support layer has a thickness of 5 to 500 μm, and a weight (weight per unit area) of 10 to 250 g/m²; and the coating layer has a thickness of 5 to 500 μm, and/or is coated in an amount of 0.1 to 200 g/m².

[6] The carrier according to any one of [1] to [5], which shows a curling height of 1.0 mm or smaller in a curling test.
[7] A transplant comprising the carrier according to any one of [1] to [6], and a cell sheet adhering to the coating layer of the carrier.
[8] A method for producing a cell sheet, which comprises the steps of:
(1) laminating the carrier according to any one of [1] to [6] on a cell sheet existing on a culture site so that the coating layer contacts with the cell sheet, and
(2) moving the cell sheet together with the carrier from the culture site.
   [9] A method for transplanting a cell sheet comprising the steps of:
(1) laminating the carrier according to any one of [1] to [6] on a cell sheet existing on a culture site so that the coating layer contacts with the cell sheet,
(2) moving the cell sheet together with the carrier from the culture site to a transplantation site,
(3) applying the cell sheet to the transplantation site, and
(4) detaching and removing the carrier by providing a non-cytotoxic condition with maintaining the cell sheet at the transplantation site.
   [10] A carrier for use in transfer of a cell sheet, which comprises a cell sheet-adhesive coating layer that can respond to a non-cytotoxic condition and a support layer consisting of a base material acceptable as a medical material.
   [11] A carrier for use in transplantation of a cell sheet, which comprises a cell sheet-adhesive coating layer that can respond to a non-cytotoxic condition and a support layer consisting of a base material acceptable as a medical material.

### Effect of the Invention

By using the carrier of the present invention, a cell sheet can be easily transferred to a transplantation site from the culture site of the cell sheet, and easily transplanted.

By using the carrier of the present invention, a cell sheet can be finely adhered to even a spherical surface such as that of eyeball.

Since the carrier of the present invention shows good detachability for the cell sheet, damage of the cell sheet can be suppressed. Therefore, it can be expected that use of the carrier of the present invention improves engraftment of the cell sheet.

### BRIEF DESCRIPTION OF THE DRAWINGS

FIG. 1 shows evaluation of a commercial product A and the carriers A to C of the present invention performed by using cell sheets. Cells were supravitally stained with a new methylene blue staining solution. First tier: Four kinds of the carriers were put on the cell sheets. Second tier: They were left at 20° C. for 5 to 10 minutes so that the cell sheets detached from petri dishes and adhered to the carriers. Third tier: The carriers were lifted up with a pair of tweezers to confirm adhesion of the cell sheets. Fourth tier: They were placed on white papers so that the cell sheets were the carriers, and the carriers were carefully peeled off.

FIG. 2 shows evaluation of the carriers B′, C′ and D of the present invention performed by using cell sheets. Cells were supravitally stained with a new methylene blue staining solution. First tier: Three kinds of the carriers were put on the cell sheets. Second tier: They were left at 20° C. for 5 to 10 minutes so that the cell sheets detached from petri dishes and adhered to the carriers. Third tier: The carriers were lifted up with a pair of tweezers to confirm adhesion of the cell sheets. Fourth tier: They were placed on white papers so that the cell sheets were under the carriers, and the carriers were carefully peeled off

FIG. 3 shows evaluation of transplantation performed by using a carrier. A cell sheet was transplanted using a carrier (carrier C of the present invention) on the cornea of affected dog as the transplantation acceptor, which had been subjected to resection of cornea surface layer under general anesthesia. First tier: Before operation (left eye). Second tier: After resection of cornea surface layer (left eye). Third and fourth tiers: Application of the cell sheet.

FIG. 4 shows infrared absorption spectra of a polylactic acid nonwoven fabric and the commercial product A.

### MODES FOR CARRYING OUT THE INVENTION

When a ratio of a component is represented with “%” in the present invention, it means weight percent, unless especially indicated.

When a range is represented as “X to Y” in the present invention, the range includes the values of X and Y as the maximum and minimum values, unless especially indicated.

### Configuration of Carrier

The present invention provides a carrier for transferring a cell sheet for transplantation comprising a cell sheet-adhesive coating layer that can respond to a non-cytotoxic condition and a support layer consisting of a base material acceptable as a medical material.

The “carrier” referred to in the present invention is a carrier used for transferring a cell sheet by being adhered to the cell sheet, unless especially indicated, and it is in the form of a membrane or sheet.

The coating layer of the carrier of the present invention mainly consists of one or more kinds of polymer materials that can respond to a non-cytotoxic condition and can adhere a cell sheet.

In the present invention, if it is described that a certain condition is “non-cytotoxic”, it is meant that the condition does not affect survivability of cell (cells) contained in a cell sheet exposed to the condition, survivability of such a cell (cells) can be restored even if the condition affects it, or the condition extremely slightly affects survivability of such a cell (cells). It can be expected that a cell (cells) exposed to a non-cytotoxic condition is engrafted at an affected part on which the cell (cells) has been transplanted, and exhibits intended functions. Whether a certain condition is a “non-cytotoxic condition” or not for a target cell can be determined by those skilled in the art in an appropriate manner.

Although specific condition of the “non-cytotoxic condition” varies depending on the type of cells to be used, the “non-cytotoxic condition” referred to in the present invention includes a temperature around body temperatures of animals, and a temperature for ordinary culture of target cell, typically a temperature of 35° C. to 42° C. Such a condition is provided by warming a transplantation site or environment to such a temperature, more specifically, by dropping a warmed isotonic solution, for example, physiological saline, cell culture medium, or buffer such as PBS.

In the present invention, the expression concerning the property of the coating layer that the coating layer “can respond” to a certain condition means that the coating layer causes such a reaction as dissolution, softening, fusion, reduction of viscosity, and decomposition, when the condition is provided, unless especially indicated.

In the present invention, the term “cell sheet-adhesive” used for the property of the coating layer means a property that the carrier can adhere a cell sheet subjected to a treatment for detachment from culture vessel surface so that the carrier and the cell sheet can be lifted up together, unless especially indicated.

The main component (component contained at a ratio higher than 50% of the solid content) of the coating layer constituting the carrier of the present invention consists of one or more kinds of components used as a viscosity enhancer, stabilizer, gelling agent, or thickener in drugs, foods, or cosmetics. According to one of the preferred embodiments, the coating layer contains, as the main component, one or more kinds of components selected from the group consisting of starch, agar, pectin, carageenan, furcellaran, alginic acid, alginate (salt or ester), galactomannan, glucomannan, tamarind gum, xanthane gum, guar gum, native type gellant gum, deacylated gellant gum, inulin, carboxymethylcellulose sodium (CMC), and gum arabic.

According to one of the preferred embodiments of the present invention, the coating layer may be constituted by a plurality of kinds of components so that the coating layer totally or partially dissolves at a non-cytotoxic temperature, the coating layer may contain additives acceptable as components of drugs, foods, cosmetics, or components for cell culture, and a component used in the coating layer may be a material subjected to a treatment for decomposition into low molecules.

According to a preferred embodiment of the present invention, the coating layer contains one or more kinds of components selected from the group consisting of starch, agar, pectin, carageenan, furcellaran, alginic acid, alginate (salt or ester), galactomannan, glucomannan, tamarind gum, xanthane gum, guar gum, native type gellant gum, deacylated gellant gum, carboxymethylcellulose sodium (CMC), and gum arabic, and another gelling agent acceptable as a component of drugs, foods, cosmetics, or a component for cell culture. According to a more specific embodiment, the coating layer contains agar, pectin, and/or starch as the main component, and contains another gelling agent acceptable as a component of drugs, foods, or cosmetics. In such an embodiment, the coating layer may contain one or more selected from the group consisting of ethylene glycol, propylene glycol, glycerol, sorbitol, mannitol, maltitol and xylitol, and may further contain a monosaccharide (for example, glucose, fructose, galactose, and xylose) and/or a disaccharide (for example, maltose, sucrose, and lactose). In addition, the inventors of the present invention confirmed that, besides the materials mentioned in the examples described in this specification, the base material can be coated with gum arabic, and it provides good detachability at a non-cytotoxic temperature. The coating layer may also contain one or more kinds selected from dispersing agent, emulsifier, stabilizer, solubilizer, colorant, preservative, processing aid, pH adjustor, and antimicrobial agent acceptable as a component of drugs, foods, or cosmetics, or as a component for cell culture, and a mixture of these. Examples of such additives include lecithin, stearates, ester derivatives of stearic acid, palmitate, ester derivatives of palmitic acid, oleate, ester derivatives of oleic acid, glycerides, ester derivatives of glycerides, sucrose polyesters, polyglycerol esters, polyoxyethylene sorbitan fatty acid esters, and polyoxyethylene alkyl ethers.

According to one of the preferred embodiments of the present invention, the coating layer contains pectin or agar having a weight average molecular weight/number average molecular weight ratio of 20 or smaller as the main component. In such an embodiment, the coating layer contains one or more kinds of additives selected from the group consisting of monosaccharides, disaccharides, oligosaccharides, dextrin, inulin, and sugar alcohols of these. When dextrin is used as an additive, it preferably has DE (Dextrose Equivalent, a value representing reducing power of a sugar solution thereof per unit amount of solid content based on that obtainable with glucose, which is taken as 100) of 18 or smaller. The ratio of weight average molecular weight/number average molecular weight of additive is preferably 20 or smaller.

It is sufficient that the coating layer used in the present invention is formed on at least one surface of the support layer, but it may be formed on both surfaces. Thickness or coating amount of the support layer can be arbitrarily determined, so long as a cell sheet adhered to the coating layer can be transferred to a transplantation site, and the carrier can be detached and removed by providing a non-cytotoxic condition with maintaining the cell sheet at the transplantation site. Typically, thickness of the coating layer may be 5 to 500 μm, for example, 10 to 100 μm. Thickness of the coating layer referred to in the present invention means thickness of one coating layer even when the carrier has the coating layers on both surface and back face, and thickness of the coating layer of the final product formed by drying after coating as required, not a thickness to be coated in the coating step, unless especially indicated. Irrespective of the thickness, the coating amount per unit area of the carrier or support layer may be 0.1 to 200 g/m², for example, 2 to 100 g/m², or 2 to 25 g/m². In addition, the support layer may consist of a nonwoven fabric as described later, and in such a case, the support layer also has a surface area as a three-dimensional structure constituted by a plurality of fibers. However, area of the carrier or support layer referred to in the present invention means an area (width) thereof as a plane, not the surface area of a three-dimensional structure, unless especially indicated.

The support layer of the carrier of the present invention consists of a base material acceptable as a medical material. The base material of the support layer is also a material on which the coating layer described above can be formed on the surface thereof. The support layer is also preferably cytocompatible.

In a preferred embodiment of the present invention, the support layer can be formed with any material selected from the group consisting of polylactic acid (PLLA), polyethylene terephthalate (PET), polyethylene (PE), polypropylene (PP), polyethylene-2,6-naphthalate (PEN), nylon 6 (N6), nylon 66 (N66), isotactic polypropylene (iPP), ethylene/tetrafluoroethylene copolymer (ETFE), tetrafluoroethylene/perfluoroalkyl vinyl ether copolymer (PFA), polyphenylene sulfide (PPS), polybutylene terephthalate (PBT), polybutylene naphthalate (PBN), polyetheretherketone (PEEK), polysulfone (P SF), polyether sulphone (PES), polyamideimide (PAI), polyetherimide (PEI), liquid crystal polymer (LCP), polychlorotrifluoroethylene (PCTFE), polyvinylidene fluoride (PVDF), polyacetal (POM), modified phenylene ether (m-PPE), acrylicnitrile/butadiene/styrene copolymer (ABS), acrylicnitrile/styrene copolymer (AS), phenol-formaldehyde (PF), urea-formaldehyde (UF), melamine-formaldehyde (MF), epoxy resin (EP), unsaturated polyester (UP), silicone resin (SI), polyglycolic acid (PGA), polylactic acid/polyglycolic acid copolymer, polycaprolactone, polybutylene succinate, polyethylene succinate, polystyrene, polycarbonate, polyhexamethylene carbonate, polyallylate, polyvinyl isocyanate, polybutyl isocyanate, polymethyl methacrylate, polyethyl methacrylate, poly(n-propyl methacrylate), poly(n-butyl methacrylate), polymethyl acrylate, polyethyl acrylate, polybutyl acrylate, polyacrylonitrile, cellulose diacetate, cellulose triacetate, methylcellulose, propylcellulose, benzylcellulose, fibroin, natural rubber, polyvinyl acetate, polyvinyl methyl ether, polyvinyl ethyl ether, polyvinyl n-propyl ether, polyvinyl isopropyl ether, polyvinyl n-butyl ether, polyvinyl isobutyl ether, polyvinyl tert-butyl ether, polyvinyl chloride, polyvinylidene chloride, poly(N-vinylpyrrolidone), poly(N-vinylcarbazol), poly(4-vinylpyridine), polyvinyl methyl ketone, polymethyl isopropenyl ketone, polyethylene oxide, polypropylene oxide, polycyclopentene oxide, polystyrene sulfone, cellulose, regenerated cellulose, promix, nylon aramide, polyvinyl alcohol, polyvinyl chloride, polyvinylidene chloride, polyurethane, polyoxymethylene, polytetrafluoroethylene, poly(p-phenylene benzobisthiazole), polyimide, and copolymers of these. The support layer may be formed from a single kind of material, or may be formed from two or more kinds of materials.

According to an especially preferred embodiment of the present invention, the support layer can be formed from, more specifically, an aliphatic polyester selected from the group consisting of polylactic acid, polyglycolic acid, polylactic acid/polyglycolic acid copolymer, polycaprolactone, polybutylene succinate, polyethylene succinate, and copolymers of these. According to a particularly preferred embodiment of the present invention, the support layer consists of polylactic acid.

Although form of the support layer used in the present invention is not particularly limited, it can be a nonwoven fabric, woven fabric, knitted fabric, or film. From the viewpoint of transfer of adhered cell sheet, the support layer preferably consists of a nonwoven fabric, which shows no directivity for strength and elongability. Although the carrier of the present invention may be explained below by exemplifying the carrier using a polylactic acid nonwoven fabric as a base material of the support layer, such explanation is also applied to the carrier using a base material other than polylactic acid nonwoven fabric, unless especially indicated.

In the present invention, when a polylactic acid nonwoven fabric is used as the base material of the support layer, fiber diameter, thickness, weight (weight per unit area), etc. thereof can be arbitrarily determined, so long as it shows appropriate plasticity etc. during use thereof as the carrier. The support layer may typically have a thickness of 5 to 500 μm, for example, 10 to 250 μm, more specifically 20 to 250 μm. Irrespective of the thickness of the support layer, the weight of the support layer can be 10 to 100 g/m², for example, 20 to 50 g/m².

In the present invention, when a polylactic acid nonwoven fabric is used as the base material of the support layer, it can be designed in consideration of tensile strength and elongability. From the viewpoint of transfer of adhered cell sheet, it can be designed so as to have a certain degree of strength. It can also be designed so as to have a certain degree of plasticity and/or a certain degree of elongability in consideration of transplantation of the cell sheet on a curved surface etc. For example, when a polylactic acid nonwoven fabric having a weight per unit area of 10 to 120 g/m² is used, it can be designed so as to have a tensile strength of 20 to 400 N, an elongability of 10 to 20%, and a tearing strength of 1.0 to 20 N.

As already described above, as the base material of the conventional carriers, there have been proposed polyvinylidene difluoride (PVDF), polypropylene, polyethylene, cellulose and derivatives thereof, papers, chitin, chitosan, collagen, and urethane resin. Further, many of the materials of the commercially available carriers were diverted from the existing food packaging materials etc. The carrier of the present invention has a special design for transferring a cell sheet, and is novel.

Therefore, it can be said that an integrated product of the carrier and a cell sheet provided by the present invention, i.e., a transplant consisting of a cell sheet adhering to the carrier, is also novel.

Size and shape of the carrier of the present invention are not limited, and various sizes and shapes can be employed in consideration of shape of cell sheet to be transferred, ease of production, ease of adhesion to a cell sheet, ease of detachment of a cell sheet, and so forth. For example, the size of the carrier of the present invention may be the same as or larger than that of a cell sheet to be transferred, and the shape may be circular shape, elliptic shape, ring shape, elliptic ring shape, fan shape, ring-like fan shape, arch shape, triangle shape, tetragonal shape, or polygonal shape. The carrier may have one or more holes. If the carrier has holes, when warmed physiological saline is dropped onto upper surface of the carrier to promote detachment of the carrier and the cell sheet upon use of the carrier, the physiological saline can pass through the carrier. Therefore, dissolution, softening, fusion, reduction of viscosity, or decomposition of the coating layer at the surface contacting with the cell sheet is more promoted. Those skilled in the art can appropriately design the shape, number, and size of the holes.

### Method for Producing Carrier

The carrier of the present invention can be produced by using conventional techniques for producing medical supplies or cell culture materials. Coating of the surface of the support layer usually comprises the step of preparing a solution of the material of the coating layer, the step of coating the base material of the support layer with the solution, and the step of forming the coating layer by gellation and/or drying.

When the coating layer comprises, as the main component, one or more kinds of materials selected from the group consisting of starch, agar, pectin, carageenan, furcellaran, alginic acid, alginate (salt or ester), galactomannan, glucomannan, tamarind gum, xanthane gum, guar gum, native type gellant gum, deacylated gellant gum, inulin, carboxymethylcellulose sodium (CMC), and gum arabic, the solution can be prepared by dissolving the aforementioned component in an aqueous solvent with warming and/or stirring if needed. Although concentration of the component in the solution can be appropriately adjusted, when agar and/or starch is used as the main component, and the coating amount per area of the support layer is 5 to 40 g/m², the concentration can be 0.3 to 15%.

When the coating layer comprises an additive acceptable as a component of drugs, foods, or cosmetics, or a component for cell culture, those skilled in the art can appropriately determine amount and concentration thereof in the solution. For example, when the coating layer contains a dispersing agent, emulsifier, stabilizer, or solubilizer, it can be used in an amount of 0.02 to 2% of starch as the main component, and it is more preferable to add and use it in an amount of 0.01 to 1%. Further, the additive can be added and used at a concentration of 0.3% or lower, more preferably 0.15% or lower, in the solution.

For the step of coating the base material of the support layer with the solution, means for coating and coating pattern are not limited so long as the solution can be coated in an amount required for adhering a cell sheet, and various existing means for coating or painting a sheet-shaped article or plane can be used. For example, various means such as those for dip coating, curtain coating, dot coating, spray coating, spiral spray coating, and summit spray coating can be used.

The method for producing the carrier of the present invention may include a sterilization step. So long as the object of the present invention is achieved, a sterilization step can be performed in any of various stages and with any of various means. For example, the raw material or intermediate may be sterilized, and in such a case, a sterile product can be obtained by sterilely performing the following steps. Alternatively, sterilization may be performed after the step of coating the base material of the support layer with the solution, the coated base material cut into an appropriate size may be sterilized, or the carrier wrapped with an appropriate wrapping material may be sterilized. As for the sterilization means, sterilization can be performed by using conventional techniques for producing medical supplies or cell culture materials. For example, there can be used existing techniques for sterilizing medical supplies or base materials for animal or human cell culture such as heat sterilization (for example, hot air sterilization, and steam sterilization), ultraviolet irradiation sterilization, gamma-ray sterilization, ethylene oxide gas (EOG) sterilization, hydrogen peroxide cold plasma sterilization, sterilization with chemical sterilant (for example, sterilization using glutaraldehyde preparation, orthophthalaldehyde preparation, hypochlorous acid preparation or peracetic acid preparation), filtration sterilization, and radio frequency sterilization. As one of particularly preferred sterilization means, ultraviolet irradiation sterilization can be mentioned. For any of the sterilization means, conditions required for sterilizing the carrier of the present invention can be appropriately designed by those skilled in the art.

### Method for Using Carrier

The carrier of the present invention can be used by the following steps of:
(1) laminating the carrier on a cell sheet existing on a culture site so that the coating layer contacts with the cell sheet to adhere the carrier to the cell sheet, and detaching and collecting the cell sheet together with the adhering carrier from a surface of a culture vessel,
(2) moving the cell sheet together with the carrier from the culture site, and
(3) applying the cell sheet adhering to the coating layer of the carrier to a transplantation site, and providing a non-cytotoxic condition to detach and remove the carrier while maintaining the cell sheet at the transplantation site.

When the cell sheet is formed by culturing cells, a culture base material of which surface is coated with a temperature-responsive polymer is preferably used for the culture (refer to Non-patent documents 1 to 6 mentioned above). Culture vessels having such a surface are also marketed. In the step of collecting the cell sheet existing at the culture site by using the carrier of the present invention, by adjusting the temperature of the culture base material on which the cell sheet is formed to a temperature not lower than the upper critical dissolution temperature of the coated polymer or not higher than the lower critical dissolution temperature of the coated polymer before or after adhering the carrier to the cell sheet, the cell sheet can be collected with less damage. It is also possible to detach the cell sheet in the culture medium in which the cells were cultured, or in another isotonic solution, and the medium or solution in which the cell sheet is detached can be chosen depending on the purpose. Typically, the medium used for the culture is once removed from the culture vessel, a comparatively small volume of fresh medium or isotonic solution is added in order to prevent drying, and then the cell sheet is detached and collected.

When the carrier is laminated on the cell sheet, it is preferred that bubbles are not contained between them. This lamination operation is usually performed with a sterilized pair of tweezers. Since the carrier of the present invention shows comparatively small curling (deformation) when it contacts with moisture (refer to the section of Examples), the operation of laminating it on a cell sheet is easy, and it is easily adhered to the cell sheet.

When a culture vessel coated with a temperature-responsive polymer is used, the culture vessel can be warmed at an appropriate temperature for several minutes, and this operation can be performed before or after lamination of the carrier, preferably after lamination of the carrier.

The cell sheet adhered to the carrier can be detached and collected from the surface of the culture vessel by slowly lifting it up from an end thereof together with the carrier. This operation is also usually performed with a sterilized pair of tweezers. With the configuration of the present invention, the carrier and the whole cell sheet can be adhered, and they can be lifted up together. Further, the cell sheet hardly detaches from the carrier during transfer of them. Furthermore, since the detached and collected cell sheet is adhered to the carrier, shrinkage, creasing, sagging, tearing, etc. of the cell sheet can be prevented.

The collected cell sheet can be moved together with the carrier from the culture site to a transplantation site or a culture site where another cell sheet is formed, and the cell sheet adhering to the carrier can be applied to the transplantation site, or laminated on the other cell sheet. When it is laminated on the other cell sheet, the aforementioned detachment and collection operations can be repeated.

After the cell sheet is applied to an affected part as the transplantation site (it may be a gel for training in laboratories), it is left standing for a while if needed, and then subjected to a non-cytotoxic condition. When the non-cytotoxic condition is a temperature around body temperatures of animals, or a temperature for ordinary culture of target cell, typically a temperature of 35° C. to 42° C., in order to subject the carrier to such a condition, for example, the transplantation site or environment is warmed to such a temperature, more specifically, warmed by dropping a warmed isotonic solution, for example, physiological saline, cell culture medium, or buffer such as PBS. Although volume of the solution to be dropped can be arbitrarily determined, it is usually such a volume that the dropped solution spreads over the whole carrier.

According to the present invention, since the carrier has the coating layer that can respond to a non-cytotoxic condition, the carrier can be easily detached while leaving the cell sheet at the affected part of the transplantation site. In addition, according to the examination of the inventors of the present invention, it can be expected that the carrier of the present invention is detached with a smaller power compared with conventional carriers, i.e., a power corresponding to about 9.9 to 20.0% of the power required for detaching the conventional carriers, even not under a non-cytotoxic condition (refer to the section of Examples). Such a method for using the carrier also constitutes a part of the present invention.

The present invention also provides a method for preparing a cell sheet. The method for preparing a cell sheet may comprise the following steps, besides the aforementioned step of using the carrier.

The cells including stem cells for forming the sheet can be collected from any of various tissues of various animals and human. Species of the supply source of the cells may be different from or the same as the species of the acceptor individual for transplantation of the cell sheet obtainable from the cells. The stem cells may also be prepared from iPS cells. Only target stem cells can be separated from the extracted cells and purified, if needed. The means used for the steps of collection, separation and purification are not particularly limited.

The culture step can be performed by, for example, inoculating the separated cells in a culture vessel, and culturing them in an appropriate medium. As the medium, any of media having various compositions developed for culture of stem cells can be used, and if needed, blood serum, various factors, antibiotics, and so forth can be added. Number of the cells to be inoculated can be appropriately adjusted according to age of the animal as the supply source of the cells, number of times of subculture of the cells, culture period, and so forth, but the cells can generally be inoculated at a cell density of 1×10³ to 1×10⁵ cells/cm².

For the culture of the cells, usual culture conditions are used. For example, culture is performed in an incubator at a temperature of 37° C. and 5% CO₂ concentration. The culture period can be appropriately adjusted depending on the number of inoculated cells (cell density), age of the animal as the supply source of the cells, intended size of the cell sheet, and so forth. The culture is usually continued until the cells become confluent in a culture dish. Size and shape of the cell sheet of the present invention are not particularly limited, and they may be chosen according to the size and shape of the transplantation site, and can be adjusted depending on the size and shape of the culture dish used for the culture.

Usually, after the culture is continued until the cells become confluent in a culture dish, the culture formed in the form of sheet is collected.

Quality of the cultured cell sheet can be controlled by confirming morphology of the contained cells and various markers.

The present invention also provides a method for transplanting a cell sheet using the carrier of the present invention. The transplantation method may comprise the following steps besides the aforementioned steps of using the carrier and/or preparing the cell sheet.

Although the method for contacting the cell sheet with an affected part (damaged area etc.) as the object of treatment is not particularly limited, the cell sheet can be contacted with the affected part by, for example, exposing the affected part with a surgical technique, and placing the cell sheet for transplantation on the exposed affected part. After the cell sheet is contacted with the affected part, the contact is maintained. The contact can be usually easily maintained, since the cell sheet adheres to the affected part with extracellular matrix existing on the surface of the cell sheet, and this adhesion is maintained.

By maintaining the contact of the cell sheet with the affected part, a tissue corresponding to the environment of the transplantation site is regenerated. This is mainly because the stem cells contained in the cell sheet differentiate into a tissue that should be originally exist at the transplantation site in response to the environment of the transplantation site or the actions of differentiation inducing factors such as cytokines derived from the transplantation acceptor individual.

The transplantation acceptor may be human or a non-human animal. The non-human animal includes experimental animals such as mouse, rat, and dog, wild animals, livestock, and companion animals.

Although the transplantation site is not particularly limited, it is especially preferably, as examples of tissue, such a tissue as corneal epithelium, skin epidermis, bone tissue, cartilage tissue, fat tissue, cardiac muscles, and smooth muscles, in view of the engraftability of the cell sheet for transplantation, and the differentiation potency of the stem cells contained in the cell sheet for transplantation. When the site of transplantation exists on an organ, the organ may be any of skin, alimentary canal, liver, heart, blood vessel, eye, nose, ear, and so forth. The present invention is preferably used as a transplantation technique for regenerating cornea, especially corneal epithelium, because of the handling property of the cell sheet or the carrier. For example, if the cell sheet adhering to the carrier is applied to the parenchyma of cornea exposed due to lost of cornea epithelium, the cell sheet can be finely applied with very few wrinkles because of the plasticity of the carrier.

When the treatment method of the present invention is used for regenerating cornea, examples of disease to which the treatment method is applied include, for example, chronic keratitis. Examples of the cause of chronic keratitis include ectopic eyelash, entropium palpebrae, keratoconjunctivitis sicca, traumatic injury, and so forth. Among these, in the cases where normal tear film can be formed, such as ectopic eyelash and entropium palpebrae, regeneration rate of cornea can be improved by the treatment method combined with an ophthalmologic surgery (surgical reintegration) appropriate for the objective disease.

### EXAMPLES

### 1. Production of Carrier (Production 1)

A carrier was produced by the following method.

### Materials:

Base material . . . Polylactic acid nonwoven fabric (weight per unit area, 30 g/m²; thickness, 0.17 mm; tensile strength, 49.0 N for longitudinal direction and 24.5 N for transverse direction; elongability, 15.0% for longitudinal direction and 15.0% for transverse direction; tearing strength, 3.9 N for longitudinal direction and 3.9 N for transverse direction)

Coating material . . . Polysaccharide mixture (starch, agar, pectin, glycerol, and emulsifier)

### Method:

(1) The polysaccharide mixture (8.3 g) and PBS (300 ml) are put into a vat, and dissolved at room temperature to obtain a polysaccharide solution.
(2) A polylactic acid nonwoven fabric is dipped in the polysaccharide solution.
(3) The dipped polylactic acid nonwoven fabric is placed on a PP film, and dried at 60° C.

By the aforementioned method, a carrier material of which coating amount of the coating material was 1.5±0.5 mg/cm² was obtained. This carrier material was cut into an appropriate size, and used as the carrier B′ of the present invention in the tests and evaluations described below.

### 2. Evaluation using imitation cell sheet (evaluation 1)

By using soymilk skin as an imitation cultured cells, materials for cell sheet carrier were screened for.

A commercially available polylactic acid nonwoven fabric (weight per unit area, 30 g/m²) having a diameter of about 30 mm was dipped into a polysaccharide solution prepared in the same manner as that described in the section of “Production of carrier” mentioned above to prepare a cell sheet carrier having a coating layer of about 20 μm (in the following table, this is indicated as “Polylactic acid nonwoven fabric +polysaccharide coating”). Further, the polysaccharide mixture film itself and the nonwoven fabric itself (not applied with polysaccharide), which were used as the raw materials, as well as a special paper (trade name, □Suihaku; 70 μm; LINTEC) and a commercially available support for cell sheet collection having a thickness of 50 μm and a diameter of about 30 mm (product name, CellShifter; CellSeed; henceforth referred to as “commercial product A”) were prepared.

PBS (0.1 ml) was dropped onto a glass plate, and a soymilk skin (containing 8% or more of soybean components, about 50 μm in thickness, about 25 mm in diameter) was placed thereon as an imitation cell sheet. The carrier was further laminated thereon, and they were left standing at 20° C. for 5 to 10 minutes. Two ends of the carrier were held with two pairs of tweezers to confirm whether the soymilk skin could be lifted up together with the carrier (adhesion property).

Then, the lifted carrier was placed on a commercially available gel for cell sheet training (CellSeed) at about 28° C. placed on a heater at 40° C., left standing for 30 seconds, and then detached from the soymilk skin to confirm whether only the carrier could be lifted up (detachability). At the time of the detachment, any particular treatment for dissolving the part of polysaccharide coating such as dropping of warmed physiological saline was not performed.

The results are shown in the following table.

TABLE 1

| | Adhesion | | Total |
|---|---|---|---|
| | property | Detachability | evaluation |
| Polylactic acid film | C | B | C |
| (charged surface) | | | |
| Polylactic acid film | C | C | C |
| (corona-treated surface) | | | |
| Polylactic acid nonwoven fabric + | B | A | B |
| polysaccharide coating | | | |
| Polysaccharide mixture film | B | B | B |
| Special paper | D | — | D |
| | (curling) | | |
| Commercial product A | C | A | C |
| | (curling) | | |

### Evaluation Criteria

Adhesion property: Whether the cell sheet carrier can be lifted up together with the imitation cell sheet
A: The whole imitation sheet adhered to the carrier, and they could be lifted up together.
B: Substantially whole imitation sheet adhered to the carrier, and they could lifted up together.
C: The imitation sheet and the carrier did not adhere to each other at a rate higher than 50%, but they could be lifted up together.
D: The imitation sheet and the carrier hardly adhered to each other, and they could not be lifted up together.

Detachability: Whether the cell sheet carrier can be removed without adhering to the imitation cell sheet
A: The cell sheet carrier could be easily removed.
B: Although the cell sheet carrier slightly adhered to the imitation cell sheet, the imitation cell sheet remained to adhere to the gel, and only the carrier could be removed.
Δ: The carrier adhered to the imitation cell sheet, and when the carrier was detached, a part of the imitation cell sheet was also lifted up. x: The carrier adhered to the imitation cell sheet, and only the carrier could not be lifted up.

### 3. Examination 1 Using Cell Sheet of Canine Adipose-Derived Mesenchymal Stem Cells (Evaluation 2)

### 1. Methods

### 1.1. Samples of Cell Sheet Carrier

The following samples were prepared. As the polylactic acid nonwoven fabric, one consisting of the same material as that used in the evaluation 1 was used, and coating with a polysaccharide mixture was performed in the same manner as that of the production 1.
Carrier A of the present invention: 50 g/m² (weight per unit area of polylactic acid nonwoven fabric)+20 μm (thickness of polysaccharide mixture coating)
Carrier B of the present invention: 30 g/m² (weight per unit area of polylactic acid nonwoven fabric)+20 μm (thickness of polysaccharide mixture coating)
Carrier C of the present invention: 20 g/m² (weight per unit area of polylactic acid nonwoven fabric)+20 μm (thickness of polysaccharide mixture coating)

The commercial product A was also used for comparison. In addition, the cell sheet carrier samples were used after being subjected to ultraviolet irradiation sterilization performed by the following method. The carrier samples cut into a circular shape having a diameter of 25 to 35 mm were irradiated with ultraviolet radiation emitted from two of ultraviolet radiation sterilization lamps (253.7 nm, GL15, 15 W, Toshiba) disposed in a draft chamber at an irradiation distance of about 50 cm for 6 hours or longer.

### 1.2. Cell Culture Conditions

Cell species: Canine adipose-derived mesenchymal stem cells (cADSCs)
Culture vessel: Petri dish having a diameter of 3.5 cm (UpCell (registered trademark), CellSeed)
Number of inoculated cells: 1.5×10⁶ cells/dish
Culture days: 3 days (so that cells become overconfluent)
Medium: α-MEM with 10% FBS, 1% PS

### 1.3. Method for Preparing Cell Sheet

1. About 1 g of fat tissue was extracted from back subcutaneous part of a healthy dog (female, 4 years old).
2. The fat tissue was treated in a 0.1% collagenase solution at 37° C. for 60 minutes to degrade the tissue.
3. A medium containing blood serum was added to terminate the enzymatic reaction.
4. The reaction mixture was centrifuged at 1200 rpm and room temperature for 5 minutes, and then the supernatant was removed.
5. The centrifuged cell aggregates were suspended in the medium, and cultured in a 75-cm² flask.
6. After 48 hours, the cells were washed with PBS to remove floating cells, and fresh culture medium was added to continue the culture.
7. The cells were subcultured 3 times until a cell sheet usable for the experiment was formed.
8. The cells (1.5×10⁶ cells) were suspended in 2 ml of the medium, and cultured for 3 days on a 3.5-cm petri dish.
9. The dish was taken out from an incubator, and the culture medium was removed.
10. The cells were supravitally stained with a new methylene blue staining solution (treatment for easy observation of the cell sheet).
11. Four kinds of carriers were put on the cell sheet.
12. The cell sheet and the carriers were left standing at 20° C. for 5 to 10 minutes so that the cell sheet detached from the petri dish, and adhered to the carriers.
13. The carriers were lifted up with a pair of tweezers to confirm adhesion of the cell sheet.
14. The carriers and the cell sheet under the carrier were placed on white paper, and the carriers were carefully detached.

### 2. Results

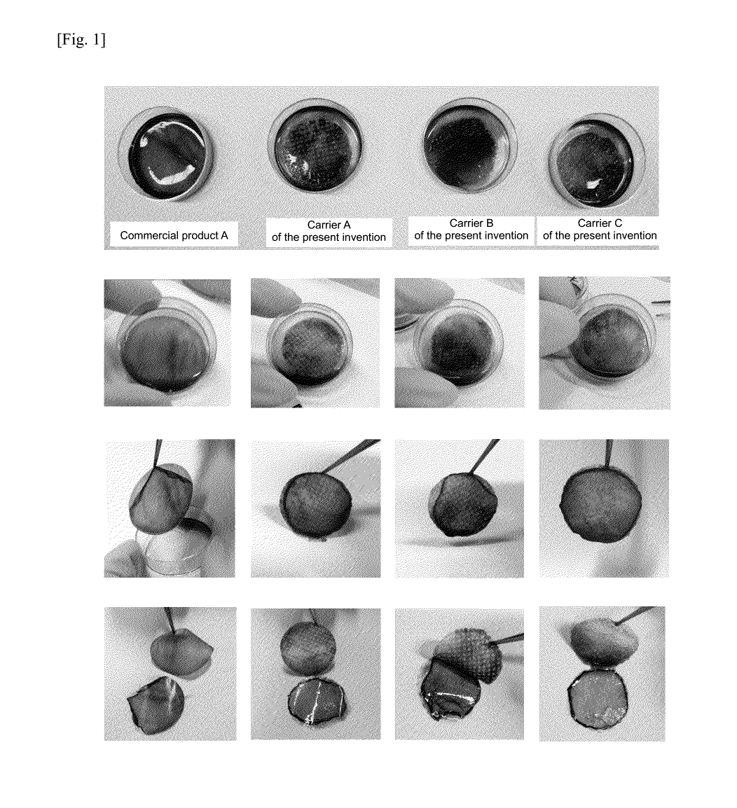

The results are shown in FIG. 1. Since the commercial product A was prepared in a size corresponding to the 3.5-cm petri dish, it could adhere the cells cultured over the whole area of the petri dish. However, since it consisted of a slippery material, slip or curling of the cell sheet was caused. However, although the sizes of the carriers A to C of the present invention did not correspond to that of the petri dish, slip or curling of the cell sheet was not observed, and they could finely adhere the cell sheet. Further, the carrier C of the present invention showed the best feeling of use, and could be easily lifted up with a pair of tweezers.

### 4. Discussion

Since the carrier C of the present invention showed superior adhesion to cells and high plasticity, and handling thereof was easy, it is considered that it can also be suitably used for transplantation of a cell sheet to the dog cornea.

### 4. Examination 2 Using Cell Sheet of Canine Adipose-Derived Mesenchymal Stem Cells (Evaluation 3)

The carriers B′, C′ and D of the present invention mentioned in the following table were prepared. For the carriers B′ and C′ of the present invention, coating with a polysaccharide mixture was performed in the same manner as that of the production 1. For the carrier D of the present invention, the sample was prepared in the same manner as that of the production 1 using a solution of pectin alone (100%), which is a thickener in the polysaccharide mixture. Further, the samples were sterilized in the same manner as that described in [3. Examination 1 using cell sheet of canine adipose-derived mesenchymal stem cells] before use.
Carrier B′ of the present invention: 30 g/m² (weight per unit area of polylactic acid nonwoven fabric)+20 μm (thickness of polysaccharide mixture coating)
Carrier C′ of the present invention: 20 g/m² (weight per unit area of polylactic acid nonwoven fabric)+20 μm (thickness of polysaccharide mixture coating)
Carrier D of the present invention: 20 g/m² (weight per unit area of polylactic acid nonwoven fabric) +20 μm (thickness of pectin coating)

TABLE 2

| | | Base material | |
|---|---|---|---|
| | | Polylactic acid nonwoven | |
| | | fabric | |
| | | Weight per | Weight per |
| | | unit area 30 | unit area 20 |
| | | g/m² | g/m² |
| Coating | Polysaccharide mixture | B′ | C′ |
| material | (same as Evaluation 1) | | |
| | Pectin | — | D |

### Polylactic Acid Nonwoven Fabric:

One having a weight per unit area of 20 g/m² had a thickness of 0.15 mm, and showed tensile strength of 29.4 N for longitudinal direction and 9.8 N for transverse direction; elongability of 15.0% for longitudinal direction and 15.0% for transverse direction; and tearing strength of 2.9 N for longitudinal direction and 2.0 N for transverse direction.

One having a weight per unit area of 30 g/m² had a thickness of 0.17 mm, and showed tensile strength of 49.0 N for longitudinal direction and 24.5 N for transverse direction; elongability of 15.0% for longitudinal direction and 15.0% for transverse direction; and tearing strength of 3.9 N for longitudinal direction and 3.9 N for transverse direction. Pectin:

Low methoxyl pectin (this had a structure consisting of a linear chain of 1,4-linked α-D-galacturonic acid polymer and functional groups such as carboxyl group and methoxy group bound to the linear chain, and showed comparatively low viscosity, in which, as for numbers of functional groups, —COOH and -COONH₂ >—COOCH₃) was used.

A cell sheet was prepared under the same conditions as those described in the section of evaluation 2, except that 2×10⁶ cells/dish were inoculated, and the culture period was one day, and adhesion property and detachability of each carrier for this cell sheet were confirmed.

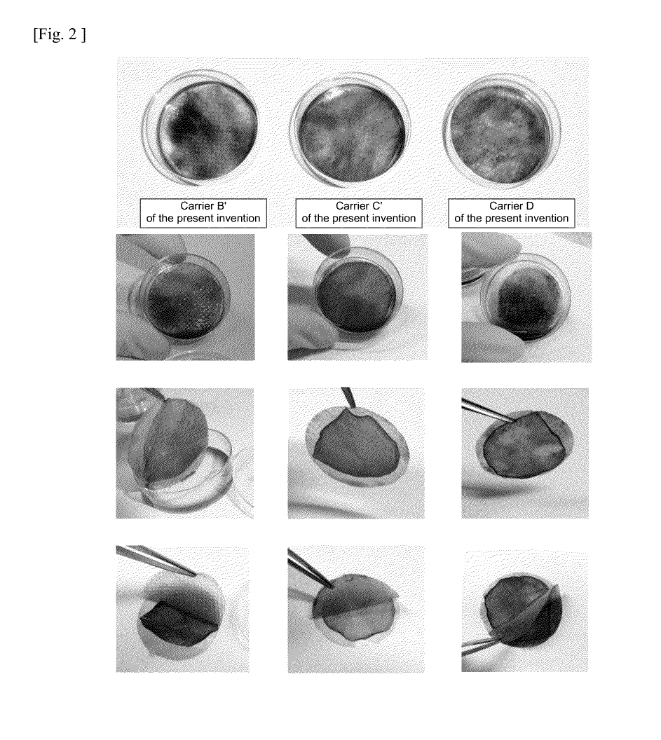

The results are shown in FIG. 2.

As a result of the examination of the carriers B′, C′ and D, it was found that all of three kinds of the carriers showed good adhesion property and detachability, like the carriers B and C examined in the evaluation 2. They showed superior adhesion property for cells, and high plasticity, and could be easily handled. Among three kinds of the carriers examined in this experiment, the carrier B′ was the most preferred in view of handling property and the conditions of the cell sheet.

### Evaluation 4: Evaluation of Transplantation Using Cell Sheet of Canine Adipose-Derived Mesenchymal Stem Cells

Cell transplantation was performed at injured part of cornea of a dog by using a cell sheet carrier.

The affected dog as the object of the transplantation of cell sheet (12 years old, pug) had chronic corneal pigmentation resulting from keratoconjunctivitis sicca, and was subjected to resection of corneal surface layer under general anesthesia before cell sheet transplantation.

As the carrier, the carrier C of the present invention mentioned in the evaluation 2 was used. A cell sheet was prepared under the same conditions as those described in the section of evaluation 2, except that 1.5×10⁶ cells/dish were inoculated, and the culture period was two days, and adhered to the carrier.

The following steps were performed following the step 12 of the evaluation 2.
13. On the cornea of the affected dog as the object of the transplantation, which had been subjected to resection of corneal surface layer under general anesthesia, the cell sheet was put together with the carrier in a state that the cell sheet was under the carrier.
14. After 5 to 10 minutes, physiological saline at 42° C. was dropped onto the carrier to promote detachment of the carrier from the cell sheet.
15. The carrier was slowly removed from the cell sheet.

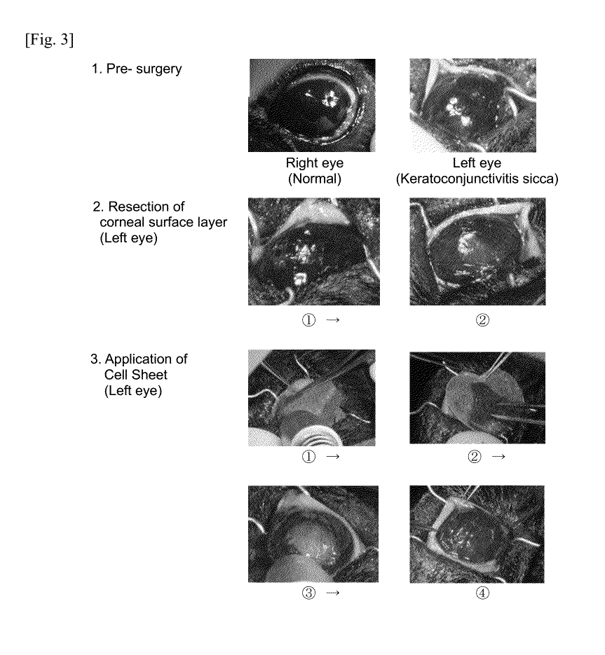

The results are shown in FIG. 3.

Since commercially available supports for collecting cell sheet consist of a slippery and comparatively hard material, detachment of them from a cell sheet may be difficult. However, since the carrier used in this experiment showed superior adhesion property for cells, and high plasticity, and could be easily handled, the operation of transplanting the cell sheet on the dog cornea could be successfully performed. In particular, the cell sheet was easily released at the target transplantation site (cornea) by warming using physiological saline at about 40° C., and transplantation could be more easily attained compared with such transplantation performed by the conventional techniques. Further, since the carrier had plasticity, the cell sheet could be adhered to the cornea having a curved surface without wrinkles at the time of the transplantation of the cell sheet.

### 5. Curling Test

Whether the cell sheet showed curling (deformation) or not at the time of contact with moisture and degree thereof were examined

### Material and Experimental Instruments:

Carrier B′ of the present invention (prepared above and cut into a circular shape having a diameter of 30 mm)
Commercially available support for collection of cell sheet having a thickness of 50 μm and a diameter of 30 mm (CellShifter, CellSeed, henceforth referred to as “commercial product A”)
PBS (phosphate buffered saline)
Digimatic Height Gage HDS-20C (Mitutoyo)

### Method:

PBS (0.05 ml) is dropped onto a glass plate. A carrier material is put thereon, and left standing for 10 seconds, and height h of curling is measured with the height gage. The measurement is performed at room temperature (see drawing below)

### Test Results:

TABLE 3

| | h: Curling height (mm) | |
|---|---|---|
| | Carrier B′ of the present invention | Commercial product A |
| 1 | 0.38 | 7.53 |
| 2 | 0.47 | 7.82 |
| 3 | 0.30 | 7.36 |
| 4 | 0.52 | 7.62 |
| 5 | 0.44 | 7.18 |
| Average | 0.42 | 7.50 |

The carrier B′ of the present invention showed little curling resulting from absorption of moisture. It is considered that when the carrier B′ of the present invention is used as a cell sheet carrier, it shows good adhesion property for a cell sheet.

### 6. Peel Strength Test

Supposing operations after cell sheet transplantation, how much power was required for detaching the carrier from the cell sheet was confirmed.

### Material and Experimental Instruments:

Carrier B′ of the present invention (prepared above and cut into a circular shape having a diameter of 30 mm)
Commercial product A
Digital Force Gauge Meter (FG-5000A, FUSORIKA)
Double-sided tape (double-coated pressure sensitive adhesive tape, No. 500, 15 mm in width, NITTO DENKO)

### Experimental Method:

(1) The double-sided tape (15 mm in width) was stuck on a glass plate, and a carrier material (30 mm in diameter) was placed thereon, so that a center part of the carrier material having a width of 15 mm was adhered.
(2) An end of the carrier material was attached to the digital force gauge meter, and lifted up in the vertical direction at a rate of 10 mm/minute, and the maximum value indicated on the measurement instrument was read (see drawing below).

### Measurement Results:

TABLE 4

| | Peel strength (mg/mm) | |
|---|---|---|
| | Carrier B′ of the present invention | Commercial product A |
| 1 | 10967 | 61267 |
| 2 | 7233 | 72833 |
| 3 | 10200 | 68600 |
| 4 | 15367 | 73133 |
| 5 | 16333 | 78467 |
| Average | 12020 | 70860 |

The carrier B′ of the present invention could be detached with a small power corresponding to about 9.9 to 20.0% of the power required for detaching the commercial product A. In addition, it was also attempted to perform the same experiment with the imitation cell sheet used in the evaluation 1 and the cell sheet used in the evaluation 2 by placing the carrier B′ of the present invention on each cell sheet, dropping physiological saline at 42° C. onto the carrier to promote detachment of the carrier from the cell sheet, and measuring peel strength in the same manner. However, since the carrier was detached with an extremely small power, the measurement could not be performed by this method.

### 7. Reference Evaluation

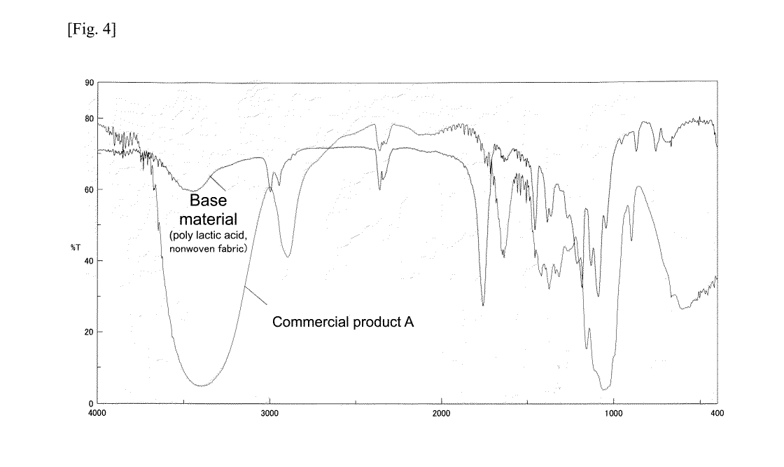

Infrared absorption spectra of the commercial product A and the polylactic acid nonwoven fabric were obtained (FIG. 4). It was thought that the material of the commercial product A was not polylactic acid, but a cellulose type material. Instrument used: FT/IR-420 Infrared Spectrometer, JASCO

### 8. Preparation Example of Carrier Using Woven Fabric

A carrier of which support layer consists of a woven fabric can be prepared by the following method.

### Materials:

Base material . . . Commercially available cotton absorbent gauze for medical use (Japanese pharmacopoeia Medical absorbent gauze standard type I; number of line per 1 cm, 12 in average for warp and 12 in average for weft; standard mass, 10.3 g for width 30 cm×length 100 cm)

Coating material . . . Polysaccharide (starch, agar, or pectin)

### Method:

(1) The polysaccharide is dissolved in 20 to 60-fold volume of water with warming as required to obtain a polysaccharide solution.
(2) The gauze is dipped into the polysaccharide solution.
(3) The dipped gauze is placed on a PP film, and dried at 60° C.

By the aforementioned method, a carrier material having a polysaccharide coating amount of 1.0 to 3.0 mg/cm² can be obtained. This material is cut into an appropriate size to obtain a carrier for cell sheet transfer.

For the obtained carrier, superior adhesion property and detachability can be confirmed by the aforementioned test using the imitation cell sheet, curling test, and peel strength test. Further, the obtained carrier can be used for transferring a cell sheet, like the aforementioned carriers consisting of a polylactic acid nonwoven fabric coated with polysaccharide.

### 9. Preparation Example of Carrier Using Film

A carrier using a film as the support layer can be prepared by the following method.

### Materials:

Base material . . . Biaxially stretched polypropylene film (Torayfan (registered trademark) 2500H; Toray Industries; thickness, 60 μm; tensile strength, 150 MPa for longitudinal direction and 300 MPa for transverse direction; elongability, 200% for longitudinal direction and 50% for transverse direction; Young's modulus, 1.8 GPa for longitudinal direction and 3.3 GPa for transverse direction)

Covering material . . . Polysaccharide mixture (mixture of starch, agar, and pectin)

### Method:

The polysaccharide mixture is dissolved in 10 to 50-fold volume of water with warming as required to obtain a polysaccharide solution. A coating layer is formed by slot-die coating so as to have a thickness of 5 to 40 μm after drying. This material is cut into an appropriate size to obtain a carrier for cell sheet transfer.

The carrier may have one or more holes. If the carrier has holes, physiological saline can pass through it when warmed physiological saline is dropped onto upper surface of the carrier, thus dissolution, softening, fusion, reduction of viscosity, or decomposition of the coating layer at the surface with which the cell sheet contacts is promoted, and detachment of the carrier and the cell sheet may become easier.

For the obtained carrier, superior adhesion property and detachability can be confirmed by the aforementioned test using the imitation cell sheet, curling test, and peel strength test. Further, the obtained carrier can be used for transferring a cell sheet, like the aforementioned carriers consisting of a polylactic acid nonwoven fabric coated with polysaccharide.

1. A carrier for transferring a cell sheet for transplantation comprising:
a cell sheet-adhesive coating layer comprising a polysaccharide that can be dissolved, softened, fused, or decomposed, or can show decrease of viscosity at a non-cytotoxic temperature, and
a support layer having the coating layer on a surface thereof and consisting of a nonwoven fabric, woven fabric, knitted fabric, or film of a base material acceptable as a medical material, wherein:
by applying a cell sheet adhering to the coating layer of the carrier to a transplantation site and providing a non-cytotoxic temperature, the carrier can be detached and removed with maintaining the cell sheet at the transplantation site.

2. The carrier according to claim 1, wherein the support layer consists of a nonwoven fabric or woven fabric.

3. The carrier according to claim 1, wherein the non-cytotoxic temperature is provided by dropping an isotonic solution at a non-cytotoxic temperature.

4. The carrier according to claim 1, wherein:
the support layer has a thickness of 5 to 500 μm, and a weight of 10 to 250 g/m²; and
the coating layer has a thickness of 5 to 500 μm, and/or is coated in an amount of 0.1 to 200 g/m².

5. The carrier according to claim 1, which shows a curling height of 1.0 mm or smaller in a curling test.

6. A transplant comprising the carrier according to claim 1, and a cell sheet adhering to the coating layer of the carrier.

7. A method for producing a cell sheet, which comprises the steps of:
(1) laminating the carrier according to claim 1 on a cell sheet existing on a culture site so that the coating layer contacts with the cell sheet, and
(2) moving the cell sheet together with the carrier from the culture site.

8. A method for transplanting a cell sheet comprising the steps of:
(1) laminating the carrier according to claim 1 on a cell sheet existing on a culture site so that the coating layer contacts with the cell sheet,
(2) moving the cell sheet together with the carrier from the culture site to a transplantation site,
(3) applying the cell sheet to the transplantation site, and
(4) detaching and removing the carrier by providing a non-cytotoxic temperature with maintaining the cell sheet at the transplantation site (implementation in a human individual is excluded).

9. A carrier for use in transfer of a cell sheet, which comprises:
a cell sheet-adhesive coating layer comprising a polysaccharide that can be dissolved, softened, fused, or decomposed, or can show decrease of viscosity at a non-cytotoxic temperature, and
a support layer having the coating layer on a surface thereof and consisting of a nonwoven fabric of a base material acceptable as a medical material.

10. A carrier for use in transplantation of a cell sheet, which comprises:
a cell sheet-adhesive coating layer comprising a polysaccharide that can be dissolved, softened, fused, or decomposed, or can show decrease of viscosity at a non-cytotoxic temperature, and
a support layer having the coating layer on a surface thereof and consisting of a nonwoven fabric of a base material acceptable as a medical material.
